(19) United States
(12) Patent Application Publication
(10) Pub. No.: US 2021/0310036 A1
(43) Pub. Date: Oct. 7, 2021

(54) METHOD OF DETECTING BACTERIUM OF GENUS HELICOBACTER USING ABSORBENT SWAB AND APPLICATION THEREOF

(71) Applicant: AJOU UNIVERSITY INDUSTRY-ACADEMIC COOPERATION FOUNDATION, Suwon-si (KR)

(72) Inventors: Kee Myung Lee, Suwon-si (KR); Choong Kyun Noh, Seoul (KR)

(73) Assignee: AJOU UNIVERSITY INDUSTRY-ACADEMIC COOPERATION FOUNDATION, Suwon-si (KR)

(21) Appl. No.: 16/837,544

(22) Filed: Apr. 1, 2020

(51) Int. Cl. C12Q 1/04; G01N 33/52; G01N 33/62; C12Q 1/58

(52) U.S. Cl. CPC C12Q 1/04; C12Q 1/58; G01N 33/62; G01N 33/526

(57) ABSTRACT

A method of detecting a bacterium of the genus Helicobacter, which includes i) inserting an absorbent swab into the stomach of a subject; and ii) absorbing gastric mucus into the absorbent swab of Step i) and separating the absorbent swab from the subject is disclosed. Also disclosed is a kit for detecting a bacterium of the genus Helicobacter, which includes an absorbent swab for absorbing gastric mucus. The method and kit, employing an absorbent swab, improves sensitivity or positive predictive value of a urease test method for detecting a bacterium of the genus Helicobacter, such as Helicobacter pylori, may significantly increase and avoids a side effect such as bleeding which may occur during tissue collection of conventional method.

### BACKGROUND

### 1. Field of the Invention

The present invention relates to a method of detecting a bacterium of the genus Helicobacter, which includes i) inserting an absorbent swab into the stomach of a subject; and ii) absorbing gastric mucus into the absorbent swab of Step i) and separating the absorbent swab from the subject.

The present invention also relates to a kit for detecting a bacterium of the genus Helicobacter, which includes an absorbent swab for absorbing gastric mucus.

### 2. Discussion of Related Art

Helicobacter pylori (H. pylori) is a gram-negative, helical-shaped bacterium found in the stomach of animals, including humans. H. pylori is a non-invasive bacterium that do not invade stomach epithelial cells, but causes damage to the stomach epithelial cells through several mechanisms, and thus is known to cause acute or chronic gastritis, stomach ulcers, duodenal ulcers, stomach cancer or MALT lymphoma. In addition, H. pylori may cause idiopathic thrombocytopenic purpura and pediatric iron deficiency anemia.

While bacteria cannot live for a long time in the stomach which is strongly acidic (pH 1 to 2) due to gastric acid, H. pylori may survive because it lives in the mucus layer, not the gastric cavity. This is because H. pylori is protected from gastric acid due to the mucus layer and lyses urea by inherently secreting urease, thereby generating ammonia and neutralizing gastric acid.

A method of detecting or diagnosing H. pylori may be roughly divided into an invasive method and a non-invasive method. The invasive method is a method directly using gastric mucosal tissue obtained by an endoscope, for example, histology, a rapid urease test, bacterial culture, or a molecular biological test method using PCR. The non-invasive method is a urea breath test, serology, a stool antigen test, or a salivary and urine antibody assay.

A rapid urease test is the most widely used among the invasive methods, and it is a method of confirming H. pylori infection due to a pH change by collecting gastric mucosal tissue and putting the tissue into an examination tool containing urea. This method utilizes the pH change due to ammonia generated by H. pylori, and has a sensitivity of approximately 85 to 98%, and a specificity of approximately 90% or more. This method is relatively simple and cheap, compared with histology, and can detect a bacterial infection quickly.

However, since the size of biopsy tissue generally collected for a rapid urease test along during endoscopy is 2 to 3 mm, a very small amount of the mucus layer is included. As described above, since H. pylori is present in the mucus layer, even in a state of infection by H. pylori, if the number of H. pylori included in the collected mucus layer is small, the result of the rapid urease test may be false negative. Particularly, like atrophic gastritis and intestinal metaplasia, when H. pylori is not uniformly distributed in the gastric mucosa, the test result is more likely to be false negative. In addition, there is a possibility of blood contained in a specimen during tissue collection or a false positive result obtained due to xylocaine or bacterial infections in the oral cavity.

It has been recommended that, to increase the accuracy of the rapid urease test, multiple types of tissue collected from various sites are used, or either an antibiotic or a proton pump inhibitor is stopped for at least two weeks prior to testing for a patient scheduled for a rapid urease test. In addition, in Korean Unexamined Patent Application No. 2000-0033013, a rapid urease test kit, which includes HCl-KCl buffer and an indicator showing a color change at pH 3.0 to 5.0, and is capable of rapidly detecting H. pylori and preventing the infection of other bacteria is disclosed.

However, there is no study or report on improvement in a sample used in a rapid urease test, and there is a lack of countermeasures against side effects such as excessive bleeding, perforations, or infections, which can occur during the collection of stomach tissue.

### SUMMARY OF THE INVENTION

Therefore, the inventors had attempted to provide a method for improving the accuracy of a urease test method and minimizing side effects that can occur in tissue collection. As a result, they had confirmed that, when an absorbent swab selectively absorbing and isolating gastric mucus is used as a sample for the urease test method, the accuracy of the urease test method can be significantly improved and the collection of gastric mucosal tissue may be excluded, and thus the present invention was completed.

Accordingly, the present invention is directed to providing a method of detecting a bacterium of the genus Helicobacter, which includes: i) inserting an absorbent swab into the stomach of a subject; and

ii) absorbing gastric mucus into the absorbent swab of Step i) and separating the absorbent swab from the subject.

The present invention is also directed to providing a kit for detecting a bacterium of the genus Helicobacter, which includes an absorbent swab for absorbing gastric mucus.

To attain the above-mentioned objects, the present invention provides a method of detecting a bacterium of the genus Helicobacter, which includes i) inserting an absorbent swab into the stomach of a subject; and

ii) absorbing gastric mucus into the absorbent swab of Step i) and separating the absorbent swab from the subject.

According to an exemplary embodiment of the present invention, the absorbent swab may be prepared in the form of any one or more selected from the group consisting of thread, cotton, woven fabric, knitted fabric and non-woven fabric.

According to an exemplary embodiment of the present invention, the insertion in Step i) may be performed using biopsy forceps.

According to an exemplary embodiment of the present invention, the absorption in Step ii) may be selective absorption of gastric mucus so that gastric tissue is not attached to the absorbent swab.

According to an exemplary embodiment of the present invention, the detection method may further include iii) inserting the absorbent swab separated in Step ii) into an examination tool for a urease test method.

According to an exemplary embodiment of the present invention, the bacterium of the genus Helicobacter may be H. pylori.

The present invention provides a kit for detecting a bacterium of the genus Helicobacter, which includes an absorbent swab for absorbing gastric mucus.

According to an exemplary embodiment of the present invention, the absorption may be selectively absorbing gastric mucus so that gastric tissue is not attached to the absorbent swab.

According to an exemplary embodiment of the present invention, the absorbent swab may be prepared in the form of any one or more selected from the group consisting of thread, cotton, woven fabric, knitted fabric and non-woven fabric.

According to an exemplary embodiment of the present invention, the bacterium of the genus Helicobacter may be H. pylori.

According to an exemplary embodiment of the present invention, the detection may be performed using a urease test method.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a diagram comparing a conventional biopsy sampling method and a sweeping method;

FIG. 2 shows the absorption of gastric mucus on the surface of the gastric mucosa into an absorbent swab using biopsy forceps;

FIG. 3 shows a urease test kit into which an absorbent swab absorbing gastric mucus is put; and

FIGS. 4A and 4B show the time for detecting H. pylori according to each of the sweeping method and the conventional biopsy sampling method.

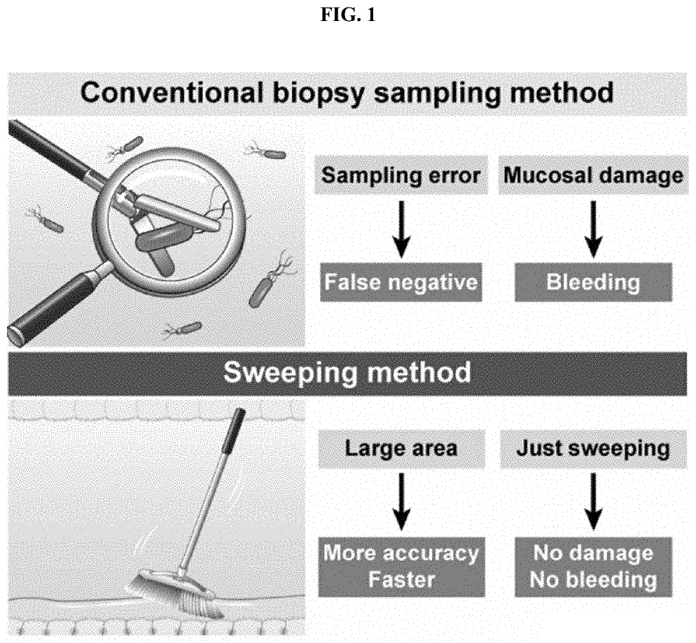

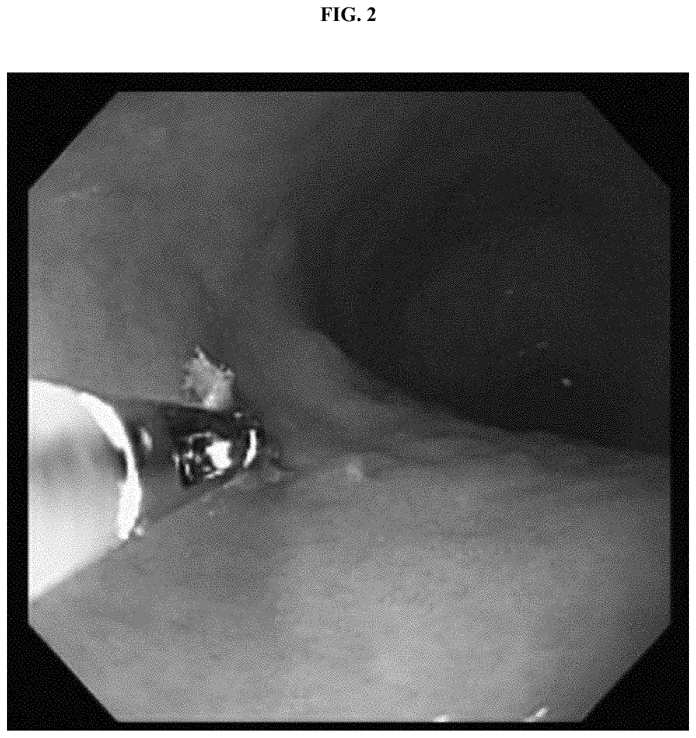

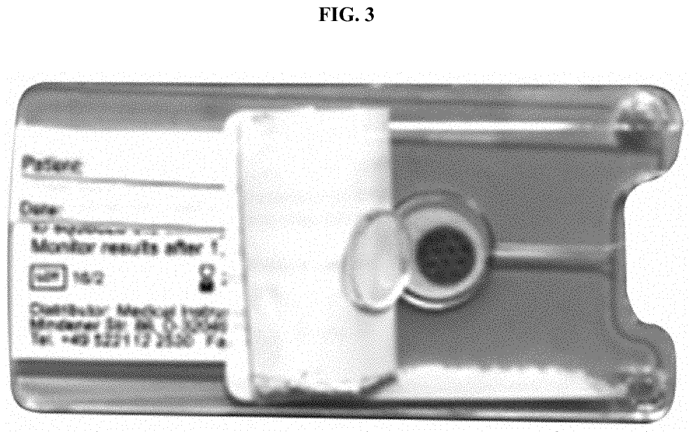

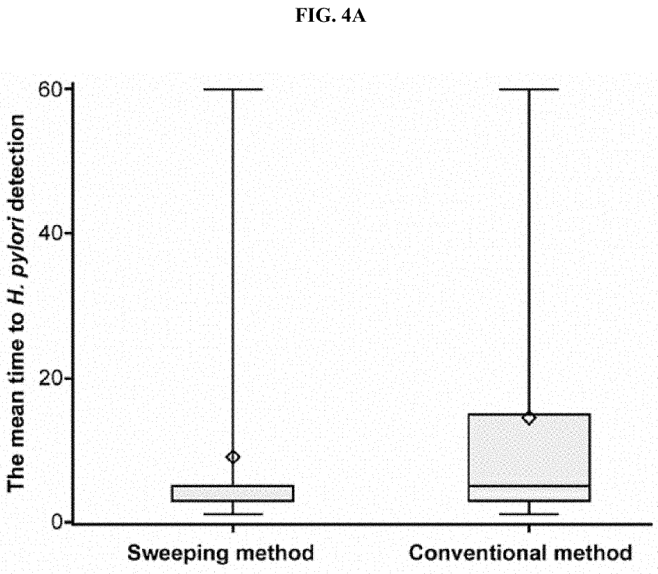

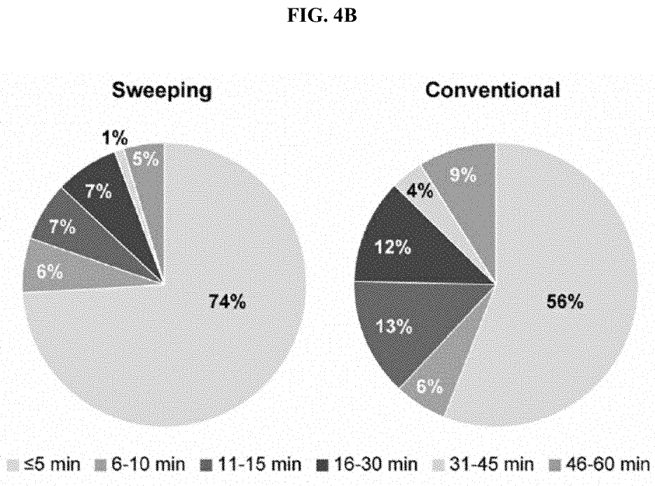

### DETAILED DESCRIPTION OF EXEMPLARY EMBODIMENTS

Hereinafter, terms of the present invention will be described.

The “urease test method” used herein refers to any method that can be used in detection or diagnosis of H. pylori using a pH change caused by ammonia produced by urea secreted by H. pylori.

The “examination tool for a urease test method” refers to a tool or kit used in a urease test method, which can be used to detect or diagnose H. pylori by inducing or detecting a pH change due to ammonia generated by urease secreted by H. pylori.

The “sweeping method” refers to a method of selectively absorbing and isolating the mucus layer in the stomach using an absorbent swab of the present invention to be used as a sample for a conventional urease test method or examination tool.

Hereinafter, the present invention will be described in detail.

As described above, in the conventional urease test method, since the collection of gastric mucosal tissue is essential, there are problems of a side effect such as bleeding and decreased detecting and diagnostic accuracy due to bacteria of the genus Helicobacter living only in the gastric mucus of the gastric mucosa. To overcome these shortcomings, the development of a sample that can increase the sensitivity of a urease test method is required, but effective sample preparation has not been studied yet.

The present invention may provide an effective method or kit for detecting a bacterium of the genus Helicobacter by selectively absorbing gastric mucus into an absorbent swab rather than gastric tissue to be used as a sample for a urease test, thereby significantly increasing the accuracy of the diagnosis or detection of a bacterium of the genus Helicobacter such as H. pylori.

Accordingly, the present invention may provide a method of detecting a bacterium of the genus Helicobacter, which includes: i) inserting an absorbent swab into the stomach of a subject; and

ii) absorbing gastric mucus into the absorbent swab of Step i) and separating the absorbent swab from the subject.

The absorbent swab of the present invention is preferably a material which has high absorbency for gastric mucus or the mucus layer or on which the gastric mucus may be easily smeared on its surface. Since the surface of the gastric mucosa has to be swept to collect the gastric mucus, the absorbent swab should not be easily torn apart, and since damage to the gastric mucosa has to be minimized during use, the absorbent swab should be neither rough nor hard. When being inserted into the stomach using biopsy forceps, the absorbent swab has to pass through an inner channel, and therefore, it should be able to fold flexibly in the channel. The absorbent swab may be any material satisfying the above-described conditions without limitation, and is preferably a fiber.

The fiber is the umbrella term for highly absorbent fibers such as polyethylene or polyester microfibers or composite microfibers of a polyester and nylon, which are 1.0 denier or less, and the fiber is preferably a polyester fiber which is easily formed into a microfiber by spinning and division, but is not limited thereto. The fiber may be any known product commercially available from leading domestic and foreign manufacturers.

The fiber refers to a general one-dimensional, two-dimensional, or three-dimensional fiber structure, wherein the one-dimensional fiber structure is thread, filament yarn, staple fiber, cotton yarn or rod-type fiber, the two-dimensional fiber structure is woven fabric, knitted fabric, non-woven fabric or a sheet, and the three-dimensional fiber structure is an elastic bandage, net, a thermoforming product or cotton, and may also include modules or final products obtained by combination with a different material. The form of the fiber may be any one or more selected from the group consisting of thread, cotton, woven fabric, knitted fabric and non-woven fabric, but the present invention is not limited thereto. The non-woven fabric refers to a type of fiber structure obtained by arranging fibers in a parallel or non-directional manner without a weaving process and combining the fibers by mechanical entanglement between the fibers, addition of a resin adhesive, heat fusion or the formation of a chemical complex. The woven fabric refers to a fiber structure manufactured by interweaving fibers as warp and weft, and the knitted fabric refers to a fiber structure manufactured by forming loops with one fiber, and these fabrics may be manufactured by various applicable methods.

Since the absorbent swab is able to be used with a conventional examination tool for a urease test method, it preferably has a size that is able to be inserted into a sample input space of the examination tool such as a kit. This is to allow the mucus and a reagent to react quickly and effectively by making maximum contact with the reagent contained in the input space. To do so, the diameter of the absorbent swab is preferably 1 to 10 mm, more preferably, 3 to 8 mm, and most preferably, 5 to 6 mm. The weight of the absorbent swab is preferably 10⁻⁶ to 1.0 g, more preferably, 10⁻⁵ to 10⁻¹ g, and most preferably, 10⁻⁴ to 10⁻² g or less. In the insertion of a sample for examination, a piece of a taping paper that has been attached to the sample input space of the test tool of the kit is detached to expose the sample input space, the sample is inserted, and then the taping paper has to be reattached. To this end, the thickness of the absorbent swab is preferably smaller than the depth of the sample input space in the kit, that is, 0.5 to 5 mm, more preferably, 1 to 4 mm, and most preferably, 1 to 2 mm.

The absorbent swab was sterilized at the sterilization level for a medical endoscope, that is, the highest level of sterilization using an ethylene oxide (EO) gas sterilization method. An EO gas sterilization method is a sterilization method for inhibiting or chemically inhibiting the reproduction of cells by penetrating the cell wall of bacteria. The absorbent swab of the present invention was prepared by vacuum-packaging following sterilization in a central sterilization room.

In the present invention, to verify a diagnostic rate of a sweeping method according to a region of the stomach, different absorbent swabs were used for the antrum and the corpus, but when the infection of H. pylori is confirmed using the detection method of the present invention, the mucus may be collected from various regions of the stomach using one absorbent swab. Unlike the conventional rapid urease test method or histology, which used gastric tissue collected from a limited region, the sweeping method of the present invention does not need gastric tissue collection, and thus there is no limitation on the number of times of mucus detection and a region of the stomach from which mucus will be collected. In consideration that bacteria of the genus Helicobacter are not uniformly distributed in the stomach but concentrated in a specific region, it can be seen that the sweeping method of the present invention is significantly improved, compared with a conventional method of detecting a bacterium of the genus Helicobacter in which tissue was collected only from a limited stomach region. The sweeping number and region may be adjusted according to the tester's judgment.

The subject is preferably a human who needs to confirm whether a bacterium of the genus Helicobacter is present in his/her stomach, but the preset invention is not limited thereto. The subject may be an animal including a human, but preferably, a mammal including a human or a bird.

A method of inserting the absorbent swab into the stomach of a subject may be any method of absorbing gastric mucus and separating the absorbent swab from the subject without limitation. For example, the insertion of the absorbent swab is preferably performed using biopsy forceps, but the present invention is not limited thereto. The cup size of the biopsy forceps is preferably 1 to 10 mm, more preferably, 1.5 to 7 mm, and most preferably, 2 to 4 mm. The type of biopsy forceps is preferably any one selected from the group consisting of fenestrated forceps, ellipsoid forceps, jumbo forceps, fenestrated forceps with needle and alligator forceps. When using biopsy forceps, the diameter of a diagnostic endoscope channel through which the biopsy forceps pass is approximately 2 to 2.8 mm.

When the gastric mucus is absorbed into the absorbent swab, it is preferable to selectively absorb gastric mucus so that gastric mucosal tissue is not attached to the absorbent swab. This is because gastric mucosal tissue is not needed since the purpose of the present invention is to detect a bacterium of the genus Helicobacter living in the mucus layer of the stomach. However, although the gastric mucosal tissue naturally detaching, not artificially scraped, may be unintentionally attached to the absorbent swab, this does not affect the effects of the present invention. In addition, since the bacteria of the genus Helicobacter are not uniformly distributed in the gastric mucosa but may be locally distributed, it is preferable to absorb the gastric mucus by moving the absorbent swab to sweep as much of the gastric mucous membrane as possible.

Preferably, the detection method of the present invention further includes iii) inserting the absorbent swab separated in Step ii) into a test tool for a urease test method.

It is known that bacteria of the genus Helicobacter live in some types of birds, as well as mammals including a human, and among these bacteria, the most famous one is H. pylori. Since the bacteria of the genus Helicobacter mass-produce urease, they may propagate in an animal stomach which is strongly acidic.

The urease test method is a detection or diagnostic method utilizing the characteristic of a bacterium of the genus Helicobacter producing urease, and the higher the number of bacteria of the genus Helicobacter in a test sample, the higher the accuracy. The urease test method is preferably any one or more selected from the group consisting of a CLO® test kit, a HpFast® test kit, a PyloriTek® test kit, a rapid urease test kit, an ultrarapid urease test kit, an urease reagent strip test kit, a PyloPlus® test kit and an Accutest® test kit, but any test method using a pH or color change caused by urease generated by a bacterium of the genus Helicobacter may be used without limitation.

The detection method of the present invention may be used to detect bacteria belonging to the genus Helicobacter producing urease without limitation, and preferably to be used to detect H. pylori, but the present invention is not limited thereto.

The present invention may also provide a kit for detecting a bacterium of the genus Helicobacter, which includes an absorbent swab for absorbing gastric mucus.

The absorption, absorbent swab, bacteria of the genus Helicobacter and urease test method according to the present invention are the same as described and used in the method of detecting a bacterium of the genus Helicobacter, and thus descriptions thereof are replaced by the above descriptions.

The kit refers to a commercially available kit generally used in the detection of a bacterium of the genus Helicobacter, and is preferably any one or more selected from the group consisting of a kit for a CLO test, which is the above-described urease test method, a HpFast® test kit, a PyloriTek® test kit, a rapid urease test kit, an ultrarapid urease test kit, an urease reagent strip test kit, a PyloPlus® test kit and an Accutest® test kit, but any test method using a pH or color change caused by urease generated by a bacterium of the genus Helicobacter may be used without limitation.

Hereinafter, the present invention will be described in further detail with reference to examples. The examples are merely provided to more fully describe the present invention, and it will be obvious to those of ordinary skill in the art that the scope of the present invention is not limited to the following examples.

### Example 1

Target Patients

Target patients underwent an upper endoscopy from June 2018 to January 2019 and required or requested a H. pylori test. Exclusion criteria included pregnancy, a history of H. pylori eradication, under 20 years of age, recent use of an antibiotic and probiotics (within the last 2 months), a contraindication to biopsy due to severe coagulopathy and commitment to participate in research. Written consent was received from each patient on the day of the procedure. General demographic information and details on past medical histories, drugs (therapeutic agent for ulcer, including an antibiotic, an antiplatelet and a PPI) and reasons for the RUT were recorded. In addition, patients took a PPI within 2 weeks before endoscopy and were subjected to subgroup analysis.

A total of 279 patients were enrolled in this research, and all patients were subjected to four H. pylori tests. The average age of the patients was 59.76 years, and 69.2% of the patients were males. 243 (87.1%) of the 279 patients had an ulcer, cancer, mucosa associated lymphoid tissue lymphoma or an adenoma. Collectively, atrophy or metastasis was confirmed by histopathology in 245 patients (87.8%).

### Example 2

Method of Detecting H. pylori

<2-1> Endoscopy

An upper endoscopy was performed using a 1-channel endoscope (Olympus Q260J; Olympus Optical Co., Tokyo, Japan) by four specialist gastroenterologists. Standard biopsy forceps with a 6-mm opening diameter were used for all patients (FB-21 K-1; Olympus, Tokyo, Japan).

Four methods for detecting H. pylori (a sweeping method of the present invention, a conventional biopsy sampling method, histopathological confirmation including immunohistochemistry (IHC) staining, and RT-PCR of paraffin-embedded tissue) were performed during the same endoscopic procedure, and gastric mucosal regions from which samples were obtained were not allowed to overlap. For a rapid urease test (RUT), a commercially available campylobacter-like organism (CLO) kit (PyloPlus; Gulf Coast Scientific, FL, USA) was used. After a sample was loaded in a kit, a color change from yellow to red within 60 minutes at room temperature as suggested by the manufacturer, indicated positive RUT. For analysis, a color change using a color matching method in the test kit was evaluated for the first 15 minutes and then every minute for 5 minutes up to 60 minutes. The total detection time was defined as the shortest time to obtain the result, and detection times for the antrum and the corpus were measured separately. The amount of gastric fluid obtained during the upper endoscopy was categorized as four levels as follows: minimal, small, intermediate and large. Bleeding was evaluated as no bleeding, minimal, oozing and spurting, and damage was evaluated as no damage, superficial, submucosal exposure and muscle exposure.

<2-2> Sweeping Method

A sweeping method of the present invention is a method of grabbing an absorbent swab with forceps and wiping the mucous membrane using a sweeping motion. A polyester wafer was used as an absorbent swab since it can selectively absorb gastric mucus without damaging gastric mucosal tissue, is not harmful to the body and is elastic. A 6-mm circular piece (thickness: 2 mm, weight: 0.001 g) sterilized by an EO gas sterilization method was punched out from non-woven fabric (polyester wafer) and then placed at the end of the endoscope device. During sweeping, the gastric fluid was not absorbed. An absorbent swab was grabbed with forceps and inserted into the stomach through an endoscope channel, and then the large curvature side of the gastric antrum was swept back and forth 10 times. Afterward, the absorbent swab was put into a sample insertion well of a CLO kit for confirming a color change. The same method as described above was repeated for the gastric corpus. Although the endoscope was sterilized, before a test, the possibility of contamination through an endoscope channel was examined, and the wafer was inserted into the channel to confirm a color.

<2-3> Conventional Biopsy Sampling Method

According to a standard protocol for general sampling, gastric antrum and corpus tissue samples were collected. In the antrum, at a large curvature side and a small curvature side as the region where sweeping was not performed, samples were obtained, respectively. In the corpus, at 4 cm near the angulus and in the middle part of the large curvature side of the corpus, samples were obtained, respectively. The obtained samples were evaluated using a CLO kit.

<2-4> Biopsy and Histopathological Confirmation

Gastric mucosal tissue samples were also obtained from all patients for histopathological examination, and two tissue samples were obtained from the antrum (large curvature) and two tissue samples were obtained from the corpus (large curvature and small curvature). All samples were fixed in a 10% buffered formalin solution and embedded in paraffin, and then sent to a pathology department. The presence of H. pylori was confirmed by performing hematoxylin and eosin staining, Giemsa staining and immunohistochemical staining (IHC) on all samples. The results were analyzed by two pathologists who did not know the results of the sweeping method and the convention biopsy sampling method.

<2-5> Genomic DNA Extraction and RT-PCR

Formalin-fixed paraffin-embedded tissue blocks of gastric biopsy specimens were retrieved from the archives of the pathology department. To obtain a sufficient amount of genomic DNA, 7-8 sections with a thickness of 10 μm were cut from the formalin-fixed paraffin-embedded tissue block. After deparaffination and rehydration, DNA extraction was performed using a QIAmp174® DNA micro kit (Qiagen, Hilden, Germany) according to the manufacturer's instructions. To confirm the infection caused by H. pylori, a U-TOP™ HPy ClaR detection kit (SeaSun Biomaterials, Daejeon, Republic of Korea) was used. RT-PCR was performed using a CFX96 RT-PCR detection system (Bio-Rad, Hercules, Calif., USA) according to the manufacturer's manual. Data was analyzed using Bio-Rad CFX manager v1.6 software (Bio-Rad, Hercules, Calif., USA). The presence of H. pylori was determined by a fluorescent signal of a detection probe and a corresponding melting temperature.

### Example 3

Result of H. pylori Detection

<3-1> Detection of H. pylori Infection by Gold Standard

The inventors defined a state of H. pylori infection as positive if at least one of the histopathology (IHC) and PCR results was positive according to the method of detecting H. pylori according to Examples 2-4 and 2-5 were positive. All of the other cases were considered uninfected.

<3-2> State of H. pylori Infection

Overall, 187 (67.0%) patients tested positive for H. pylori infection using the gold standard definition. The remaining 92 (33.0%) patients tested negative for H. pylori, of which a negative result was obtained on all four tests in 69 (75.0%) patients. The sweeping method detected not only 176 (63.1%) cases but also 16 (17.4%) of the gold standard-negative cases. H. pylori-positive rates were as follows: sweeping method, 68.8%; conventional method, 50.5%; histology, 64.2%; and PCR, 63.1%. The H. pylori-positive rate for the sweeping method was significantly higher than that for the conventional, histology, or PCR method (P<0.001).

<3-3> Diagnostic Performance of Sweeping Method for H. pylori Detection

TABLE 1

| | Rapid urease test | |
|---|---|---|
| Characteristic (95% CI) | Sweeping | Conventional |
| Sensitivity | 0.941 (0.897-0.970) | 0.685 (0.613-0.750) |
| Specificity | 0.826 (0.733-0.897) | 0.859 (0.771-0.923) |
| Accuracy | 0.903 (0.862-0.935) | 0.742 (0.686-0.792) |
| PPV | 0.917 (0.868-0.952) | 0.908 (0.848-0.950) |
| NPV | 0.874 (0.785-0.935) | 0.573 (0.486-0.656) |

As shown in Table 1, the sensitivity of the sweeping method was 0.941 (95% CI, 0.897-0.970), which was higher than that for the conventional method at 0.685 (95% CI, 0.613-0.750). The specificity of the sweeping was 0.826 (95% CI, 0.733-0.897) versus 0.859 (95% CI, 0.771-0.923) for the conventional method. The overall accuracy rate of H. pylori detection for the sweeping method was 0.903 (95% CI, 0.862-0.935) versus 0.742 (95% CI, 0.686-0.792) for the conventional method. Therefore, the sweeping method has higher sensitivity, accuracy, PPV, and NPV than the conventional method for H. pylori detection.

<3-4> Time for Positive Results in Sweeping Method and Conventional Method

TABLE 2

| | Sensitivity | Specificity | Accuracy | PPV | NPV | Kappa value | | |
|---|---|---|---|---|---|---|---|---|
| Time | (%) | (%) | (%) | (%) | (%) | Conventional | Histology | PCR |
| ≤5 min | 0.971 | 0.882 | 0.935 | 0.925 | 0.954 | 0.535 | 0.835 | 0.794 |
| (n = 232) | | | | | | | | |
| ≤15 min | 0.975 | 0.872 | 0.937 | 0.929 | 0.954 | 0.492 | 0.844 | 0.781 |
| (n = 254) | | | | | | | | |
| ≤30 min | 0.977 | 0.872 | 0.940 | 0.934 | 0.954 | 0.468 | 0.848 | 0.780 |
| (n = 268) | | | | | | | | |
| ≤60 min | 0.978 | 0.872 | 0.943 | 0.938 | 0.954 | 0.470 | 0.851 | 0.785 |
| (n = 279) | | | | | | | | |

As shown in Table 2, an average time until H. pylori detection for the sweeping method was 9.12±13.62 minutes, which was faster than that of the conventional method (14.58±17.48 minutes, P=0.003). In the results of the sweeping method, among all of the H. pylori positive cases, 155 cases (155/193, 80.3%) showed a time of less than 10 minutes, and 143 cases (143/193, 74.3%) showed a time of less than 5 minutes. The results of the sweeping method were consistent at all time points: <5, <15, <30 and <60 min. In addition, the sweeping method result, the IHC staining result (total kappa value, 0.851) and the PCR result (total kappa value, 0.785) were highly consistent.

### Example 4

Study Results

<4-1> Side Effects

TABLE 3

| Side effect | Sweeping | | Conventional | | P-value |
|---|---|---|---|---|---|
| Bleeding, n(%) | | | | | <.001 |
| none | 271 | (97.1) | 37 | (13.3) | |
| minimal | 5 | (1.8) | 210 | (75.3) | |
| oozing | 3 | (1.1) | 30 | (10.8) | |
| spurting | | | 2 | (0.7) | |
| Damage, n(%) | | | | | |
| none | 273 | (97.9) | | | <.001 |
| superficial | 6 | (2.2) | 235 | (84.2) | |
| submucosa exposure | | | 33 | (11.8) | |
| muscle exposure | | | 11 | (3.9) | |

As shown in Table 3, oozing occurred in 3 patients (1.1%) according to the sweeping method, whereas oozing occurred in 30 patients (10.8%) and spurting occurred in 2 patients according to the conventional method. Six cases of superficial damage (2.2%) were observed according to the sweeping method, whereas 33 cases of submucosal exposure (11.8%) and 11 cases of muscle exposure (3.9%) were observed according to the conventional method. Tissue damage due to the removal of parenchymal tissue was inevitable.

<4-2> Performance Characteristics for Various Conditions

Diagnostic performance of the sweeping and conventional methods with respect to various conditions such as a tissue sample acquisition location (antrum/corpus), atrophy with or without metastasis, PPI use, and a peptic ulcer or stomach cancer was analyzed. Under all conditions, the sensitivity and accuracy of the sweeping method were higher than those of the conventional method. Particularly, in ulcer or stomach cancer patients whose accurate diagnosis is important, the sweeping method had high sensitivity, specificity, accuracy, PPV and NPV (Sensitivity: probability of being positive when a patient with infection is tested, Specificity: probability of being negative when a patient without infection is tested, Positive predictive value (PPV): probability of a patient being genuinely infected when the test is positive for the patient who may or may not have an infection, Negative predictive value (NPV): probability of a patient not being genuinely infected when the test is negative for the patient who may or may not have an infection, Accuracy: probability of determining an (genuine) infection tested positive as positive, and an infection tested negative as negative, Sweeping: the result using the sweeping method of the present invention, and Conventional: the result using conventional CLO test).

[Statistical Analysis]

A sample size was calculated to detect a 0.132 difference between two diagnosis tests in which the sensitivity was 0.985 or 0.853, respectively, with 90% statistical power. This procedure uses a two-sided McNemar test with a significance level of 0.05. The prevalence of H. pylori infection in the general population is 0.530. The ratio of mismatched pairs is 0.185. Based on the sample size calculation, at least 200 participants were needed. The time to detect H. pylori was compared between the conventional method and the sweeping method using a t-test, and mucosal bleeding and the extent of damage were compared using Fisher's direct verification. By including a 95% confidence interval (CI), sensitivity, specificity, accuracy, a positive predictive value (PPV) and a negative predictive value (NPV) were calculated. Cohen's kappa coefficient was used to determine the consistency between the sweeping method and the conventional method, histopathology or PCR. The kappa value was able to be interpreted as follows: <0.00, zero; 0.00-0.20, slight: 0.21-0.40, fair; 0.41-0.60, moderate; 0.61-0.80, substantial; 0.81-1.00, almost perfect. A receiver operating characteristic curve corresponding to 95% CI was calculated, and a significant difference in an area under the receiver operating characteristic (AUROC) region between two methods was calculated. All reported P-values were obtained by a bilateral test, p values<0.05 were considered significant. Analyses were performed with SAS version 9.4 (SAS Institute Inc., Cary, N.C., USA).

Therefore, when a method or kit for detecting a bacterium of the genus Helicobacter using an absorbent swab of the present invention is used, the sensitivity or positive predictive value of a urease test method for detecting a bacterium of the genus Helicobacter such as H. pylori significantly increases, so that a more accurate result of detecting or diagnosing a bacterium of the genus Helicobacter can be provided. In addition, since gastric mucosal tissue, which has been required for a conventional urease test method, is no longer needed, side effects, for example, bleeding that may occur in tissue collection, may not occur, and therefore, it is more effective as a method of detecting or diagnosing a bacterium of the genus Helicobacter.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A method of detecting a bacterium of the genus Helicobacter, comprising:
i) inserting an absorbent swab into the stomach of a subject; and
ii) absorbing gastric mucus into the absorbent swab of Step i) and separating the absorbent swab from the subject.

2. The method of claim 1, wherein the absorbent swab is any one or more selected from the group consisting of thread, cotton, woven fabric, knitted fabric and non-woven fabric.

3. The method of claim 1, wherein the insertion of Step i) is performed using biopsy forceps.

4. The method of claim 1, wherein, in the absorption of Step ii), gastric mucus is selectively absorbed such that gastric tissue is not attached to an absorbent swab.

5. The method of claim 1, further comprising:
iii) inserting the absorbent swab separated in Step ii) into a test tool for a urease test method.

6. The method of claim 1, wherein the bacterium of the genus Helicobacter is Helicobacter pylori.

7. A kit for detecting a bacterium of the genus Helicobacter, comprising an absorbent swab for absorbing gastric mucus.

8. The kit of claim 7, wherein the absorption is selectively absorbing gastric mucus such that gastric tissue is not attached to the absorbent swab.

9. The kit of claim 7, wherein the absorbent swab is any one or more selected from the group consisting of thread, cotton, woven fabric, knitted fabric and non-woven fabric.

10. The kit of claim 7, wherein the bacterium of the genus Helicobacter is Helicobacter pylori.

11. The kit of claim 7, wherein the detection is performed using a urease test method.
